(19) United States
(12) Patent Application Publication
(10) Pub. No.: US 2024/0385091 A1
(43) Pub. Date: Nov. 21, 2024

(54) METHOD AND DEVICE FOR CAPTURING AND DETECTING A SAMPLE COMPONENT

(71) Applicant: InnoTech Alberta Inc., Edmonton (CA)

(72) Inventors: Xiujie LI, Edmonton (CA); Earl JENSON, Vegreville (CA); Wei LI, Edmonton (CA)

(21) Appl. No.: 18/664,062

(22) Filed: May 14, 2024

Related U.S. Application Data
(60) Provisional application No. 63/467,446, filed on May 18, 2023.

(51) Int. Cl. G01N 1/40; G01N 1/14; G01N 27/02; G01N 33/00; G06T 7/00

(52) U.S. Cl. CPC G01N 1/4077; G01N 1/14; G01N 27/02; G01N 33/0098; G06T 7/0012; G06T 2207/30188

(57) ABSTRACT

The present disclosure provides a method of detecting the presence of and/or the amount of at least one fungal plant pathogen in a sample. The method comprises contacting the at least one fungal plant pathogen with at least one biological component that specifically binds to the at least one fungal plant pathogen in a liquid sample, detecting, such as imaging and/or measuring the impedance of, at least a portion of the liquid sample, and correlating the detection with the presence and/or the amount of the at least one fungal plant pathogen in the liquid sample. The present disclosure also provides a sample trap device for performing the method.

### FIELD

The present disclosure relates to a method and device for capturing and detecting a plant pathogen in a sample.

### BACKGROUND

The following paragraphs are not an admission that anything discussed in them is prior art or part of the knowledge of persons skilled in the art.

Plant diseases are a global concern, having a significant impact on agricultural productivity and food security with regard to access, availability, utilization and stability. Over 80 percent of plant diseases are caused by fungi, which can be unicellular or multicellular. The most well-known plant diseases caused by fungal pathogens are fusarium head blight of wheat and barley, black stem rust and leaf rust of wheat, sclerotinia stem rot and blackleg of canola, leaf blight of corn, ergot of sorghum, late blight of potato, fusarium wilts of cotton and flax, powdery and downy mildews of grape, etc. Most fungi reproduce via spores, which allow fungi to spread from one plant to another, and from one location to another. These spores may be moved by wind, insects, or water.

The primary tool used to control the infection of plants by fungal pathogens is the application of fungicides. Fungicides are most effective when applied before infection occurs or during a key stage of infection, that is, early flowering and before the appearance of symptoms in the crops. There are two groups of fungicides: protectant and systemic fungicides. Protectant fungicides kill fungal spores upon contact and prevent infection from occurring. However, protectant fungicides naturally decrease over time due to weathering, such as degradation by sunlight or heat, and due to redistribution over the plant surface by rainfall, dew or irrigation water. Systemic fungicides, on the other hand, are absorbed by the plant and are less susceptible to weather-related issues. However, systematic application of fungicides can be unprofitable as the outbreak of plant infections can vary greatly among fields and years.

### INTRODUCTION

The following introduction is intended to introduce the reader to this specification but not to define any invention. One or more inventions may reside in a combination or sub-combination of the instrument elements or method steps described below or in other parts of this document. The inventors do not waive or disclaim their rights to any invention or inventions disclosed in this specification merely by not describing such other invention or inventions in the claims.

The infection of plants by fungal pathogens primarily occurs via airborne spores, which can spread over a large area. Airborne ascospores, conidiospores, aeciospores and urediniospores are the dominant source of the spread of infection in plants. Methods and devices that can directly capture and detect airborne ascospores and/or conidiospores may provide a more efficient measurement of the risk of crop infection.

A spore trap captures spores for identification and/or quantification. A typical spore trap consists of an approximately flat surface where spores are passively deposited. Although such traps are economical, they are not a reliable method for the collection of spores as the positioning of the trap can be altered by external factors, such as wind or rain, which impact spore deposition. Furthermore, these spore traps may attract contaminants, such as leaves, that may interfere with spore quantification.

Quantitative real-time polymerase chain reaction (qPCR) has been developed as the method of choice for monitoring airborne ascospores by amplifying a selected segment of their DNA for detection and quantification. Although qPCR has the sensitivity and selectivity to detect the presence of pathogens to a level as low as a single spore in the sample, it has a number of disadvantages in terms of cost and complexity of the method due to the simultaneous requirements of thermal cycling and fluorescence detection which may render the technique unsuitable for routine onsite field applications.

There remains a need for methods and devices for capturing and detecting fungal plant pathogens that are less costly, more efficient, and/or more capable of onsite field application.

The present disclosure provides a method and a sample trap device for securely capturing and selectively moving spores for detection. The presently disclosed invention may be: 1) more efficient in detecting the presence of and/or the amount of spores; 2) more effective in detecting the presence of and/or the amount of spores, by, for example, decreasing contaminants in a sample and/or increasing detection accuracy by concentration; and/or 3) less costly by, for example, using more cost efficient sensor parts such as antibodies and chips, compared to one or more known spore traps or methods of trapping spores.

The present disclosure provides a method of detecting the presence of and/or the amount of at least one fungal plant pathogen in a sample. The method comprises contacting the at least one fungal plant pathogen with at least one biological component that specifically binds to the at least one fungal plant pathogen in a liquid sample, detecting, such as imaging and/or measuring the impedance of, at least a portion of the liquid sample, and correlating the detection with the presence and/or the amount of the at least one fungal plant pathogen in the liquid sample.

Optionally, the method comprises concentrating the liquid sample before detecting the liquid sample. Optionally, the concentrating is performed by attracting the at least one biological component bound to the at least one fungal plant pathogen to a portion of the sample. Optionally, the biological component is an antibody or fragment thereof conjugated to a magnetic bead. Optionally, the attracting is performed using a magnet or magnetizable member switchable between an activated magnetic field state and a deactivated magnetic field state.

Optionally, the method comprises sending the liquid sample to an image capturing device, for example, a camera or a microscope. Optionally, the method comprises concentrating the liquid sample within the field of view of the image capturing device. Optionally, the concentrating is performed using a magnet or magnetizable member switchable between an activated magnetic field state and a deactivated magnetic field state.

Optionally, the method comprises biosensing at least a portion of the liquid sample. Optionally, biosensing comprises measuring the impedance of at least a portion of the liquid sample. Optionally, measuring the impedance comprises: measuring the impedance of the at least a portion of the liquid sample; and correlating the change of measured impedance and a reference impedance with the presence and/or the amount of the at least one fungal plant pathogen in the liquid sample. Optionally, measuring the impedance of the at least a portion of the liquid sample is performed by moving the liquid sample along a flow channel that is in fluid communication with at least two electrodes for measuring impedance.

The present disclosure also provides a sample trap device. The sample trap device comprises: at least one reservoir having at least one outlet, and at least one magnet or magnetizable member switchable between an activated magnetic field state and a deactivated magnetic field state, the magnet or magnetizable member operably coupleable to the reservoir to magnetically engage at least one component of the reservoir. The at least one outlet is in fluid communication with a detection unit. Optionally, the at least one magnet or magnetizable member is an electromagnet.

Optionally, the at least one magnet or magnetizable member is coupleable to a side wall of the reservoir.

Optionally, at least a portion of the side wall of the reservoir has an angle of inclination that is greater than about 90 degrees relative to the bottom of the reservoir. Optionally, the at least one magnet or magnetizable member is operably coupleable to magnetically engage at least one component on the portion of the side wall.

Optionally, the detection unit comprises an image capturing device, for example, a camera or microscope. Optionally, the image capturing device comprises a least one magnet or magnetizable member switchable between an activated magnetic field state and a deactivated magnetic field state, the at least one magnet or magnetizable member operably coupleable to the image capturing device to magnetically engage the at least one component.

Optionally, the detection unit comprises a biosensor. Optionally, the biosensor comprises: a support structure, at least two electrodes coupled to the support structure, and an impedance measurement circuit coupled to the at least two electrodes.

The herein disclosed sample trap device may be used to perform the herein disclosed method.

Optionally, the herein disclosed method and sample trap device are automated.

Optionally, the sample comprises at least one fungal plant pathogen that infects a canola plant, a wheat plant, a barley plant, a corn plant, a rice plant, a millet plant, a sorghum plant, or a combination thereof, or Fusarium graminearum, F.avenaceum, F. poae, F.sporotrichioedes, Puccinia spp., Puccinia triticina, P. recondite, P. striiformis, Erysiphe graminis f.sp. tritici, Glomerella graminicola (anamorph Colletotrichum graminicola), Pyrenophora tritici-repentis (telomorph) and Drechslera tritici-repentis, Pyrenophora trichostoma, Urocystis agropyri, Sclerotinia borealis, Septoria spp., Stagnospora spp. Parastagonospora nodorum, Pyrenophora spp. teres tares, Pyrenophora teres maculate, Claviceps purpurea, Alternaria spp., Heminthosporium spp, Pseudocercosporella herpotrichoides, Glomerella graminicola (anamorphic), Colletotrichum graminicola, Fusarium verticillioides, Gibberella zeae, Aspergillus flavus, A. parasiticus, Lasiodiplodia theobromae, Physoderma maydis, Exserohilum turcicum, Cochliobolus heterostrophuspp, Cercospora zeae-maydis and Cercospora zeinaor, Cochliobolus carbonum, Stenocarpella maydis, Puccinia polysora, Magnaporthe oryzae, Cochliobolus miyabeanus, Ascochyta oryzae, Drechslera gigantean, Microdochium albescens, Cercospora oryzae, Puccinia graminis f.sp. oryzae, Uromyces coronatus, Ramularia oryzae, Bipolaris setariae, Cercospora penniseti, Curvularia penniseti, Dactuliophora elongata, Drechslera dematioidea, Claviceps fusiformis, Exserohilum rostratum, Beniowskia sphaeroidea, Myrothecium roridum, Phyllosticta penicillariae, Pyricularia grisea, Puccinia substriata, Moesziomyces penicillariae, Sclerotium rolfsii, Gleocercospora sorghi, Sarocladium strictum (syns Acremonium strictum), Cephalosporium acremonium, Macrophomina phaseolina, Claviceps africana, Sphacelia sorghi, Fusarium spp. moniliforme (syn. Gibberella fujikuroi), F. thapsinum (syn. G. thapsina), Aspergillus spp., Fusarium andiyazi, F. nygamai, Penicillium spp., Cercospora sorghi, Passalora fusimaculans (syn. Cercospora fusimaculans), Setosphaeria turcica (syns. Exserohilum turcicum, Helminthosporium spp. turcicum), Periconia circinata, Ramulispora sorghicola, Gibberella fujikuroi (syns. Fusarium moniliforme var. subglutinans G. fujikuroi var. subglutinans, G. intermedia, F. proliferatum), Ascochyta sorghi, Puccinia purpurea, Sclerotium rolfsii (syn. Athelia rolfsii), Ramulispora sorghi, Phyllachora sacchari, Bipolaris sorghicola (syns. B. cookei, Helminthosporium cookei.), Gloeocercospora sorghi, or any combination thereof.

Other aspects and features of the present disclosure will become apparent to those ordinarily skilled in the art upon review of the following description of specific examples in conjunction with the accompanying figures.

### BRIEF DESCRIPTION OF THE DRAWINGS

Embodiments of the present disclosure will now be described, by way of example only, with reference to the attached Figures.

FIG. 1 is a photograph of an example of a sample trap device as disclosed herein.

FIG. 2 is a cross-sectional illustration of an example of a sample trap device as disclosed herein.

FIG. 3 is photograph of an example of a detection unit as disclosed herein.

FIG. 4 is an image of an example of antibody coated beads conjugated to spores as disclosed herein.

FIG. 5 is a photograph of an example of a biosensor as disclosed herein.

FIG. 6 is a photograph of an example of eight biosensors as disclosed herein connected together.

FIG. 7 is a photograph of an example of a biosensor as disclosed herein.

FIG. 8 is a photograph of an example of a sample trap device as disclosed herein.

FIG. 9 is a photograph of an example of a sample trap device as disclosed herein.

FIG. 10 is a circuit schematic of an example of a sample trap device as disclosed herein.

### DETAILED DESCRIPTION

Generally, the present disclosure provides a method of detecting the presence of and/or the amount of at least one fungal plant pathogen in a sample, comprising: contacting the at least one fungal plant pathogen with at least one biological component that specifically binds to the at least one fungal plant pathogen in a liquid sample; detecting, such as imaging and/or measuring the impedance of, at least a portion of the liquid sample; and correlating the detection with the presence and/or the amount of the at least one fungal plant pathogen in the liquid sample.

Detecting the presence of at least one fungal plant pathogen in a sample refers to determining whether a fungal plant pathogen is present or absent in the sample. Detecting the amount of fungal plant pathogen in the sample refers to determining how many fungal plant pathogens are present in the sample. The detecting may be performed on the entire sample, multiple portions of the sample, or one portion of the sample. The detecting may be performed while the sample or any portion thereof is approximately stationary or in motion. The detecting may be performed by any method that is capable of determining the presence and/or absence of a fungal plant pathogen in a sample and/or determining how many fungal plant pathogens are in a sample. Examples of detecting include imaging and biosensing.

The at least one fungal plant pathogen may be any pathogen that infects a crop plant, for example, a canola plant, a wheat plant, a barley plant, a corn plant, a rice plant, a millet plant, a sorghum plant, or a combination thereof.

In some examples, the at least one fungal plant pathogen infects canola plants and causes grey mold. The at least one fungal plant pathogen that infects canola plants may be Botrytis cinerea.

In some examples, the at least one fungal plant pathogen infects wheat and barley plants and causes scab, fusarium head blight, leaf rust including black, brown, and yellow rust, black stem rust, powdery mildew, canker, tan spot, flag smut, snow scald, leaf blotch, net blotch, alternaria leaf blight, ergot, black point, eyespot, or a combination thereof. The at least one fungal plant pathogen that infects wheat and barley plants may be Fusarium graminearum, F. avenaceum, F. poae, F. sporotrichioedes, Puccinia graminis, Puccinia triticina, P. recondite, P. striiformis, Erysiphe graminis f.sp. Tritici, Glomerella graminicola (anamorph Colletotrichum graminicola), Pyrenophora tritici-repentis (telomorph) and Drechslera tritici-repentis, Pyrenophora trichostoma, Urocystis agropyri, Sclerotinia borealis, Septoria spp., Stagnospora spp., Pyrenophora teres tares, Pyrenophora teres maculate, Claviceps purpurea, Alternaria spp., Heminthosporium spp, Psudocercosporella herpotrichoides, or a combination thereof.

In some examples, the at least one fungal plant pathogen infects corn plants and causes leaf blight and stalk rot, ear and kernel rot, leaf rust, black stem rust, black kernel rot, brown spot, grey leaf spot, Northern corn leaf spot, southern leaf blight, white ear rot, root and stalk rot, southern rust, or a combination thereof. The at least one fungal plant pathogen that infects corn plants may be Glomerella graminicola (anamorphic), Colletotrichum graminicola, Fusarium verticillioides, Gibberella zeae, Aspergillus flavus, A. parasiticus, Lasiodiplodia theobromae, Physoderma maydis, Exserohilum turcicum, Cochliobolus heterostrophu, Cercospora zeae-maydis and Cercospora zeinaor, Cochliobolus carbonum, Stenocarpella maydis, Puccinia polysora, or a combination thereof.

In some examples, the at least one fungal plant pathogen infects rice plants and causes rice blast, brown spot, collar rot, eyespot, leaf scald, narrow brown leaf spot, rusts, white leaf streak, or a combination thereof. The at least one fungal plant pathogen that infects rice plants may be Magnaporthe oryzae, Cochliobolus miyabeanus, Ascochyta oryzae, Drechslera gigantean, Microdochium albescens, Cercospora oryzae, Puccinia graminis f.sp. oryzae, Uromyces coronatus, Ramularia oryzae, or a combination thereof.

In some examples, the at least one fungal plant pathogen infects millet plants and causes bipolaris leaf spot, cercospora leaf spot, curvularia leaf spot, dactuliophora leaf spot, drechslera leaf spot, ergot, exserohilum leaf blight, false mildew, Head mold, myrothecium leaf spot, phyllosticta leaf blight, pyricularia leaf spot, rust, smut, southern blight, top rot, zonate leaf spot, or a combination thereof. The at least one fungal plant pathogen that infects millet plants may be Bipolaris setariae, Cercospora penniseti, Curvularia penniseti, Dactuliophora elongata, Drechslera dematioidea, Claviceps fusiformis, Exserohilum rostratum, Beniowskia sphaeroidea, Myrothecium roridum, Phyllosticta penicillariae, Pyricularia grisea, Puccinia substriata, Moesziomyces penicillariae, Sclerotium rolfsii, Fusarium moniliforme, Gleocercospora sorghi, or a combination thereof.

In some examples, the at least one fungal plant pathogen infects sorghum plants and causes acremonium wilt, ergot, uematsu, fusarium head blight, root and stalk rot, grain storage mold, gray leaf spot, ladder leaf spot, leaf blight, milo disease (periconia root rot), oval leaf spot, pokkah boeng (twisted top), rough leaf spot, rust, southern sclerotial rot, sooty stripe, tar spot, target leaf spot, zonate leaf spot and sheath blight, or a combination thereof. The at least one fungal plant pathogen that infects sorghum plants may be Sarocladium strictum (syns Acremonium strictum), Cephalosporium acremonium, Macrophomina phaseolina, Claviceps africana, Sphacelia sorghi, Fusarium spp., Fusarium moniliforme (syn. Gibberella fujikuroi), F. thapsinum (syn. G. thapsina), Aspergillus spp., Fusarium andiyazi, F. nygamai, Penicillium spp., Cercospora sorghi, Passalora fusimaculans (syn. Cercospora fusimaculans), Setosphaeria turcica (syns. Exserohilum turcicum, Helminthosporium turcicum), Periconia circinata, Ramulispora sorghicola, Gibberella fujikuroi (syns. Fusarium moniliforme var. subglutinans G. fujikuroi var. subglutinans, G. intermedia, F. proliferatum), Ascochyta sorghi, Puccinia purpurea, Sclerotium rolfsii (syn. Athelia rolfsii), Ramulispora sorghi, Phyllachora sacchari, Bipolaris sorghicola (syns. B. cookei, Helminthosporium cookei.), Gloeocercospora sorghi, or a combination thereof.

In the context of the present disclosure, reference to the fungal plant pathogen refers to its entire life cycle. For example, Sclerotinia sclerotiorum refers to the fungus, the produced sclerotium, and the produced apothecia. Sclerotinia sclerotiorum can also be known as cottony rot, watery soft rot, stem rot, drop, crown rot and blossom blight. Sclerotinia minor, for example, is a pathogen with this life cycle.

The at least one fungal plant pathogen infects a canola plant, a wheat plant, a barley plant, a corn plant, a rice plant, a millet plant, a sorghum plant, or a combination thereof. In some examples, the at least one fungal plant pathogen infects more than one type of crop plant, for example, Sclerotinia sclerotiorum.

The sample may be any medium in which one or more fungal plant pathogens may reside and be capable of being recognized by at least one biological component. In some examples, the sample may be in a fluid such as a solution or a liquid.

Contacting the at least one fungal plant pathogen with at least one biological component may comprise physically interacting, binding, and/or recognizing at least one portion of the at least one fungal plant pathogen with at least one biological component. Specifically binding to the at least one fungal plant pathogen refers to a biological component that interacts with the at least one fungal plant pathogen in close proximity or a biological component that recognizes and/or binds to the at least one fungal plant pathogen with specificity. Optionally, the at least one biological component may recognize and/or bind to more than one type of fungal plant pathogen.

The biological component may be any molecule, compound or polypeptide that is capable of physically interacting with, binding, and/or recognizing at least a portion of the fungal plant pathogen in a liquid sample. In some examples, the biological component recognizes and binds to at least one fungal plant pathogen with a dissociation constant (Kd) less than or equal to a micromolar (μM), for example, a nanomolar (nM). In some examples, the biological component recognizes and specifically binds to at least one fungal plant pathogen with a dissociation constant (Kd) less than a nanomolar (nM). The biological component may be an antibody or fragment thereof. The antibody may be a polyclonal or a monoclonal antibody. The antibody fragment thereof may be an antigen-binding fragment (Fab) or a single-chain variable fragment.

In some examples according to the present disclosure, the biological component may be an antibody or fragment thereof conjugated to a magnetic bead. Optionally, the magnetic bead may be 2.8 μm superparamagnetic Dynabeads™ M-270 Epoxy beads. A skilled person would understand how to couple the biological component to a magnetic bead, for example, based on the protocols provided by ThermoFisher Scientific (https://www.thermofisher.com/ca/en/home/references/protocols/proteins-expression-isolation-and-analysis/antibody-protocol/antibody-coupling-kit.html).

In some examples, the antibody or fragment thereof specifically binds a spore of the herein disclosed fungal plant pathogen or at least one airborne ascospore or conidiospore released from the apothecia.

A skilled person would be able to produce an antibody that specifically binds to at least one of the herein described fungal plant pathogens. Antibodies have been successfully produced in rabbits and rats against fungal spores, for example, Mycosphaerella brassicicola (Kennedy and Wakeham, 2002 New Methods for Detecting and Enumerating Fungal Spores of Plant Pathogens. Plant Protect. Sci., 38 (Special Issue 1): 38-42; incorporated by reference), Glomus monosporum (Göbel, C., Hah,. A., Hock, B., 1995. Production of polyclonal and monoclonal antibodies against hyphae from arbuscular mycorrhizal fungi. Crit Rev Biotechnol. 1995;15 (3-4):293-304; incorporated by reference), Clostridium tyrobutyricum (María Lavilla, Ruth de Luis, Celia Conesa, María D. Pérez, Miguel Calvo & Lourdes Sánchez. 2008. Production of polyclonal antibodies against spores of Clostridium tyrobutyricum, a contaminant affecting the quality of cheese: characterisation of the immunodominant protein. Food and Agricultural Immunology 19 (1):77-91; incorporated by reference), and Aflatoxigenic Molds (R. Shapira et al., Development of Polyclonal Antibodies for Detection of Aflatoxigenic Molds Involving Culture Filtrate and Chimeric Proteins Expressed in Escherichia coli. APPLIED AND ENVIRONMENTAL MICROBIOLOGY 63(3): 990-995; incorporated by reference), and Stachybotrys chartarum (Schmechel D & Lewis DM. 2001. The production of species-specific monoclonal antibodies (Mabs) against the allergenic and toxigenic fungus Stachybotrys chartarum. The FASEB Journal, Vol. 15(4), p. A662, Abstract #523.1; incorporated by reference). Production of pathogen specific antibodies and antibody production protocol references are within the purview of a skilled person with protocols available online, for example; https://www.abcepta.com/assets/pdf/Polyclonal_Antibody_Development_Protocol.pdf; https://www.thermofisher.com/ca/en/home/life-science/antibodies/custom-antibodies/custom-antibody-production/custom-polyclonal-antibody-production/custom-rabbit-polyclonal-antibody-production- protocols.html; incorporated by reference.

An example of the herein disclosed detecting comprises imaging at least a portion of the liquid sample. Imaging at least a portion of the sample refers to selectively capturing an image of the sample or any portion(s) thereof or selectively obtaining a visual representation of the sample or any portion(s) thereof. Imaging may be performed by any image capturing device, for example, a digital/optical camera or microscope. In some examples, the camera may be a Raspberry Pi camera.

An example of the herein disclosed detecting comprises using a biosensor for biosensing at least a portion of the liquid sample. Biosensing at least a portion of the sample refers to selectively detecting and measuring a biological molecule, signal, or event using a sensor. An example of biosensing is measuring impedance. Measuring the impedance of at least a portion of the sample refers to applying a current or voltage excitation perturbation in an electrochemical cell and measuring the voltage or the current response as a function of an applied excitation frequency. Impedance is a measure of the opposition to the flow of current, arising from ion diffusion, electrode kinetics, redox reactions, and molecular interactions at the electrode surface, when an alternating excitation voltage is applied to the cell. Measuring the impedance may be performed by any measurement circuit having any voltage or current source and any voltage or current analyzer connected in an electrical path in which electrons may flow.

Impedance may be measured using at least two electrodes in fluid communication with the sample, and an impedance measurement circuit coupled to the at least two electrodes. The impedance measurement circuit may comprise a potentiostat in electrical communication with the at least two electrodes. At least one of the at least two electrodes is a working electrode and at least another of the at least two electrodes is a reference electrode or a counter electrode, and the at least two electrodes are in electrical communication with one another and an impedance measurement circuit. Optionally, the at least two electrodes are interdigitated electrodes. Examples of using at least two electrodes for measuring impedance in a liquid sample are disclosed in U.S. patent application Ser. No. 16/395,062, which is incorporated by reference. Optionally, the at least two electrodes and/or the impedance measurement circuit are incorporated into a chip. Optionally, the herein disclosed biosensor may comprise more than one chip connected in series or parallel. In some examples, more than one chip may be used for the detection of more than one plant pathogen. For example, three chips connected in series (Chip A-Chip B-Chip C) may be used for the detection of three different plant pathogens. Optionally, the at least two electrodes are coupled to a support structure, such as a circuit board. Optionally, the at least two electrodes are in fluid communication with the liquid sample within a flow channel having at least one outlet and at least one inlet to accept and/or expel liquid sample from being in fluid communication with the at least two electrodes. The flow channel may be any pathway having a sufficient size to allow at least a portion of the liquid sample to flow therethrough. In some examples, the flow channel has a diameter from about 20 μm to about 200 μm, for example, about 50 μm.

Without being bound by theory, it is believed that the herein disclosed device and method may function on the resistive pulse sensing method in which electrodes are in fluid communication with an electrolyte solution, and changes in resistance are monitored in a relatively confined space between the electrodes. When a particle flows between a pair of energized electrodes, it will momentarily dislocate the solution from this region and alter the electric field generated and thus change the measured impedance. Application of an interdigital capacitor into a flow cell for detection of charged micro particles may also be used.

An impedance spectrum may be obtained by applying a current or voltage excitation perturbation in a sample and measuring the voltage or the current response as a function of the applied excitation frequency.

Correlating the image with the presence and/or the amount of the at least one fungal plant pathogen in a sample may comprise counting the number of plant pathogens or spores conjugated to the biological component and/or may comprise using a computer-assisted image processor to determine the presence and/or the amount of the at least one fungal plant pathogen in the sample relative to a control sample. In some examples where one portion or multiple portions of the sample are imaged, the number of plant pathogens or spores detected in the one or multiple portions of the sample may be extrapolated to determine the number of plant pathogens or spores in the sample. In some examples, portions of the sample may be imaged in real time, for example, while the portions are moving past the field of view of an image capturing device. Optionally, the herein disclosed imaging may be automated.

Correlating the impedance with the presence and/or the amount of the at least one fungal plant pathogen may comprise correlating the change of impedance with a standard curve of known impedances or a reference impedance of known amounts of the at least one fungal plant pathogen. The reference impedance may be the impedance measured of a liquid sample without the at least one fungal plant pathogen. The impedance may be measured by applying from about 0 VAC to about 10 VAC, for example, 0 VAC; about 1 VAC; about 2 VAC; about 3 VAC; about 4 VAC; about 5 VAC; about 6 VAC; about 7 VAC; about 8 VAC; about 9 VAC; about 10 VAC; or from any VAC defined above to any other VAC defined above, sinusoidal excitation perturbation at from about 0 V +/−24 V; for example, 0 V; about 5 V; about 10V; about 15 V; about 20 V; about 24 V; or from any V defined above to any other V defined above, DC in a frequency range from about 1 Hz to about 1 GHz. In some examples where one portion or multiple portions of the sample are measured, the number of plant pathogens or spores detected in the one or multiple portions may be extrapolated to determine the number of plant pathogens or spores in the sample. In some examples, portions of the sample may be measured in real time, for example, while the portions are moving through a flow channel in and out of fluid communication with electrodes for measuring impedance.

Optionally, the herein disclosed detecting includes more than one detecting process, for example, imaging and biosensing may be performed in series or in parallel. In some examples, a portion of the sample may be detected by imaging and another portion of the sample may be detected by biosensing. In some other examples, the sample or any portion thereof may be detected using imaging and following imaging, detecting using biosensing. Alternatively, the sample or any portion thereof may be detected using biosensing followed by imaging. In the examples comprising more than one detecting process, the correlating described herein includes separately correlating the imaging and the biosensing with the presence and/or amount of the at least one fungal plant pathogen and amalgamating said correlations, or correlating the imaging and the biosensing together. Using more than one detecting process may, for example, improve efficiency and accuracy of detection and/or correlation.

The herein disclosed contacting; imaging; biosensing; and/or correlating may be automated, for example, the contacting; imaging; and biosensing may be input for a processor configured to correlate the input and determine the presence of and/or the amount of the at least one fungal plant pathogen in the sample.

Optionally, the herein disclosed method further comprises concentrating the liquid sample before detecting at least a portion of the liquid sample. Concentrating refers to removing and/or reducing at least some liquid or diluting agent in the liquid sample, and optionally, for example, increasing the proportion of the at least one fungal plant pathogen to a portion of the sample. An example of concentrating the liquid sample includes attracting the at least one fungal plant pathogen to a portion of the sample. Attracting refers to pulling together and/or drawing near the at least one fungal plant pathogen to a portion of the sample. An example of attracting the at least one fungal plant pathogen to a portion of the sample uses a magnet or magnetizable member switchable between an activated magnetic field state and a deactivated magnetic field state that engages the at least one fungal plant pathogen, such as through a magnetic bead conjugated to the biological component bound to the at least one fungal plant pathogen. Magnetizable refers to being capable of being magnetized or already in a magnetized state. An example of a herein disclosed magnet or magnetizable member is an electromagnet. The herein disclosed magnet or magnetizable member be any magnet such as Neodymium magnet or rare earth magnet coupled with an actuator to create an on-off motion. The magnetic bead may be any magnetic particle having a size sufficient to be conjugated to the herein disclosed biological components, for example, from about 1 μm to about 4 μm; for example, about 1 μm; about 2 μm; about 3 μm; about 4 μm; or from any size defined above to any other size defined above. Examples of magnetic beads include 2.8 μm superparamagnetic Dynabeads™ M-270 Epoxy beads (Thermo Fisher Scientific). The herein disclosed concentrating may be used when, for example, increasing sensitivity of detecting the pathogen, such as imaging and/or measuring the impedance, is desired.

Optionally, the herein disclosed method further comprises washing the liquid sample before detecting at least a portion of the liquid sample. Washing refers to any form of removing at least some impurities and/or contaminants from the liquid sample, and optionally, to increasing the concentration of the at least one fungal plant pathogen in the liquid sample. An example of washing the sample includes attracting the at least one fungal plant pathogen to a portion of the sample as disclosed herein, and adding another portion of the sample and/or an acceptable washing liquid from a storage chamber or container to the portion of the sample at a sufficient rate and amount to remove some impurities and/or contaminants from the portion of the sample. Optionally, the washing may be performed with a washing liquid such as water or phosphate buffered saline (PBS). In some examples, PBS may be used to keep the sample in a neutral pH range so that the antibodies are stable and functional. Optionally, the amount of washing liquid that may be used for washing may be from about 2 mL to about 50 mL. The herein disclosed washing may be used when, for example, increasing the efficiency and/or effectiveness of detecting the pathogen, such as imaging and/or measuring the impedance, is desirable. Optionally, the herein disclosed washing may be used to adjust the level of liquid sample within the at least one reservoir to a desired level.

Optionally, the herein disclosed method further comprises recirculating the liquid within the sample. Recirculating refers to moving liquid sample throughout a system where at least a portion of the liquid is redirected back into the system. The herein disclosed recirculating may be performed throughout the herein disclosed method, for example, throughout contacting. The herein disclosed recirculating may be used when, for example, increasing the efficiency of contacting between the at least one fungal plant pathogen and the at least one biological component is desirable.

Optionally, the herein disclosed method further comprises adding liquid to the sample to adjust the level and/or amount of liquid sample to a desired level. The added liquid may be water, an acceptable sample buffer, or the herein disclosed washing liquid.

Optionally, the herein disclosed method comprises sending at least a portion of the liquid sample to a detection unit for detecting. Sending refers to selectively moving at least a portion or portions of liquid sample for detection. Selectively moving may include: 1) exerting a force on the sample, for example, by using a herein disclosed magnet or magnetizable member guide; and/or a pump or vacuum pump; and/or 2) by withstanding a force, such as one or more operable valves that when closed, restrict the gravity flow of the liquid sample.

Optionally, in the herein disclosed examples comprising imaging, the method further comprises concentrating the liquid sample within the field of view of the image capturing device. The field of view refers to an observable area visible via the image capturing device and/or a maximal area that the image capturing device can capture. An example of concentrating the liquid sample within the field of view of the image capturing device includes using a herein disclosed magnet or magnetizable member to hold or secure the at least a portion of the liquid sample in place for image detection. The herein disclosed concentrating within the field of view of the image capturing device may be used when, for example, improving the accuracy of detecting the at least one fungal plant pathogen, is desired.

Generally, the present disclosure also provides for a sample trap device, comprising: at least one reservoir having at least one outlet; and at least one magnet or magnetizable member switchable between an activated magnetic field state and a deactivated magnetic field state, the magnet or magnetizable member operably coupleable to the reservoir to magnetically engage at least one component of the reservoir, wherein the at least one outlet is in fluid communication with a detection unit. The herein disclosed sample trap device may be used to perform the herein disclosed method. The at least one component may be the herein described fungal plant pathogen optionally bound to the herein disclosed biological component and/or magnetic bead.

The at least one reservoir may be any receptacle or chamber having one or more side walls including a bottom forming an interior volume, and capable of holding liquid and/or sample. The size of the at least one reservoir may be varied provided that it has an interior volume space sufficient to hold liquid and/or sample, for example, for holding about 5 mL to about 50 mL of liquid and/or sample. The at least one reservoir may include more than one reservoir, for example, 2 reservoirs; 3 reservoirs; 4 reservoirs; or more. In some examples, a reservoir may be divided by a wall to create two or more reservoirs. Optionally, in the herein examples including two or more reservoirs, the other reservoir has an interior volume that is larger than the interior volume of the at least one reservoir. In some examples, the other reservoir with a larger interior volume may help to increase sample collection size and to may facilitate easy mixing with the antibodies. In some examples, the at least one reservoir with the smaller interior may help in reducing the amount of liquid in the sample to achieve sample concentration. Optionally, each of the reservoirs is in fluid communication with one or more other reservoirs. Optionally, in the herein examples of two or more reservoirs, the device comprises a weir located at least partially between the fluid communication path of the two or more reservoirs, for example when increasing the efficiency of concentrating a component within the reservoir upstream of the weir is desirable. The height of the weir may be varied and/or adjustable depending on the desired amount of liquid and/or sample within the one or more reservoirs. Optionally, in herein examples including a weir, at least a portion of the interior surface, for example the bottom surface, of the reservoir upstream of the weir slants away from the weir, for example when decreasing the amount of components of interest within the liquid and/or sample moving over the weir is desirable. Optionally, the at least one reservoir further comprises a breather tube. In some examples, the breather tube may function like a liquid level controller. When the liquid level is at the tube opening level, the air is blocked from getting into the liquid container, therefore no liquid will be dispensed into the at least one reservoir. When the liquid level is lower than the tube opening, air gets in the liquid container and liquid will be dispensed to the at least one reservoir.

Optionally, the at least one reservoir may have an interior forming two opposing surfaces slanting downwards towards one another and meeting along the approximate centerline of the at least one reservoir, for example when increasing the efficiency of moving components within the reservoir towards the centerline is desirable. The angle of each slanting surface may be varied, for example, the slanting surfaces may have an angle of about 5 degrees to about 45 degrees from the centerline and/or bottom of the at least one reservoir.

Optionally, the at least one reservoir has an outlet in fluid communication with an inlet of the at least one reservoir, for example when increasing the interaction of components of the liquid and/or sample within the at least one reservoir is desirable. Optionally, in the herein examples of two or more reservoirs, the at least one reservoir has an inlet or outlet in fluid communication with an outlet or inlet, respectively, of another reservoir, for example when increasing the interaction of components of the liquid and/or sample within the at least two reservoirs is desirable. The location of the herein disclosed inlet and/or outlet may be on any wall of the at least one reservoir, for example, a side wall or bottom wall, at a sufficient height to allow fluid liquid and/or sample to flow therethrough. Optionally, at least a portion of the interior surface of the at least one reservoir may be slanted in a direction towards the herein disclosed outlet for example when increasing the efficiency of moving liquid and/or sample towards the outlet is desirable. Optionally, in herein examples of two or more reservoirs including a weir, the other reservoir has an outlet located on a side wall about opposite the weir, for example when increasing the efficiency of moving liquid and/or sample from the other reservoir to the at least one reservoir is desirable. A pump, vacuum pump, and/or the gravity flow may be used to move liquid and/or sample within the at least one reservoir and/or between two or more reservoirs.

The herein disclosed at least one magnet or magnetizable member switchable between an activated magnetic field state and a deactivated magnetic field state may be operably coupleable to any side wall, including bottom, of the at least one reservoir to magnetically engage at least one component of the reservoir. To magnetically engage at least one component of the reservoir refers to magnetically interacting with at least a portion of the at least one component within the reservoir. The location and number of magnets or magnetizable members may be varied depending on the desired location or locations within the at least one reservoir the at least one component within the liquid and/or sample is to be secured or captured and/or moved. Optionally, the herein disclosed magnet or magnetizable member is automated, for example when increasing the efficiency of securing or capturing and/or moving the at least one component is desirable.

Optionally, at least a portion of the side wall of the reservoir has an angle of inclination that is greater than about 90 degrees, for example, from about 90 degrees to about 170 degrees, about 90 degrees, about 95 degrees; about 100 degrees; about 110 degrees; about 120 degrees, about 130 degrees; about 140 degrees; about 150 degrees; about 160 degrees; or about 170 degrees relative to the bottom of the reservoir. One or more of the herein disclosed magnet or magnetizable members may be located to magnetically engage at least one component within the liquid and/or sample on the portion of the side wall, for example when increasing the separation of the at least one component from the remaining contents of the liquid and/or sample is desirable. Optionally, the at least one reservoir has an outlet located on the portion of the side wall, for example when washing the at least one component is desirable. The outlet may be in fluid communication with another portion of the at least one reservoir to wash using liquid and/or sample, and/or the outlet may be in fluid communication with a liquid storage chamber or container to wash using an acceptable washing liquid. In some examples, the acceptable washing liquid may be water and/or phosphate buffered saline.

The at least one outlet in fluid communication with the herein disclosed detection unit may be located at any portion of the side wall including bottom of the at least one reservoir. Optionally, in the herein examples including at least a portion of the side wall of the reservoir having an angle of inclination, the at least one outlet is located at the portion of the side wall or below the portion of the side wall, for example when increasing the concentration of the component being moved to the detection unit is desirable.

Optionally, in the herein examples where the detection unit comprises the herein disclosed image capturing device, the image capturing device comprises a herein disclosed magnet or magnetizable member to engage the at least one component within the field of view of the image capturing device. Optionally, the image capturing device and/or the at least one magnet or magnetizable member of the image capturing device is automated, for example when increasing the efficiency of imaging is desirable.

Optionally, in the herein examples where the detection unit comprises the herein disclosed biosensor, the components of the biosensor are automated, for example when increasing the efficiency of imaging is desirable.

The herein disclosed automation refers to components of the herein disclosed method or device being in communication with a processor configured to perform a herein disclosed method or function and/or a herein disclosed device feature. The herein disclosed components of automation may be comprised within an automation system. For example, the processor may be configured to switch the herein disclosed magnet(s) or magnetizable member(s) between an activated magnetic field state and a deactivated magnetic field state; operate pumps and/or valves to control the movement of liquid and/or sample to and from the at least one reservoir, the detection unit, and/or between two or more reservoirs; and/or moving washing liquid from a liquid storage chamber or container to the at least one reservoir. Optionally, the processor is in electrical communication with a modem for wireless control and/or transmission.

Optionally, the herein disclosed sample trap device comprises a roof and/or weather shield to protect the at least one reservoir and/or the content therein, from the weather, such as rain, hail, and/or unwanted particles. Optionally, the herein disclosed sample trap device comprises a net covering at least a portion of the opening of the at least one reservoir to decrease contaminants such as animals and/or debris from entering into the at least one reservoir.

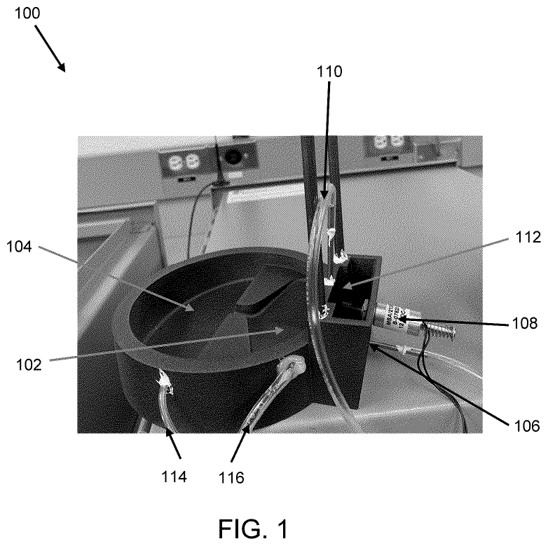

FIG. 1 illustrates an example of a sample trap device as herein described. The sample trap device (100) comprises: two reservoirs (102, 104) having an outlet (106), wherein the two reservoirs (102, 104) are in fluid communication with one another. Reservoir (104) has an interior volume of about 50 mL and reservoir (102) has an interior volume of about 10 mL. The device further comprises a magnet or magnetizable member (108) switchable between an activated magnetic field state and a deactivated magnetic field state. The magnet or magnetizable member (108) is operably coupleable to a side wall of the reservoir to magnetically engage at least one component within the reservoir. The outlet (106) is in fluid communication with a detection unit (not shown). The sample trap device further comprises another inlet (110) located at the portion of the side wall (112) of the reservoir (102) that has an angle of inclination that is about 97 degrees relative to the bottom of the reservoir. The sample trap device further comprises an inlet and outlet (114, 116) for recirculating the liquid sample.

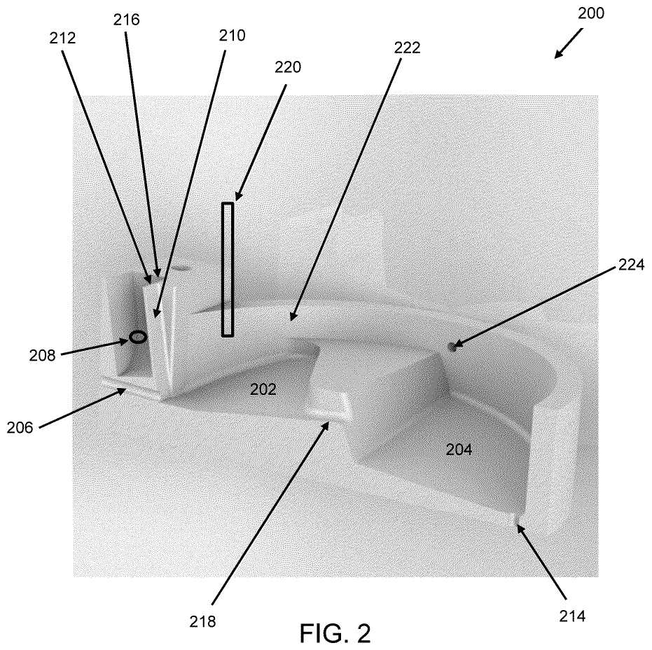

FIG. 2 is a cross-sectional illustration of an example of a sample trap device as described herein. The sample trap device (200) comprises: two reservoirs (202, 204) having an outlet (206), wherein the two reservoirs (202, 204) are in fluid communication with one another. Reservoir (204) has an interior volume of about 50 mL and reservoir (202) has an interior volume of about 10 mL. The device further comprises a magnet or magnetizable member (208) switchable between an activated magnetic field state and a deactivated magnetic field state. The magnet or magnetizable member (208) is operably coupleable to a side wall (210) of the reservoir to magnetically engage at least one component within the reservoir. The outlet (206) is in fluid communication with a detection unit (not shown). The detection unit (not shown) is in fluid communication with inlet (224) to return sample from detection to the reservoir. The sample trap device further comprises another inlet (212) located at the portion of the side wall (210) of the reservoir that has an angle of inclination that is about 97 degrees relative to the bottom of the reservoir. The sample trap device further comprises an outlet and inlet (214, 216) for recirculating the liquid sample. Optionally, (212) and (216) may be combined into the same inlet. The sample trap device further comprises a weir (218) located at least partially between the fluid communication path of the two reservoirs (202, 204). The interior surface at the bottom of reservoir (202) slants downwards away from the weir (218). The interior surface at the bottom of reservoir (204) slants downwards away from the weir (218) towards the outlet (214). The sample trap device further comprises a breather tube (220), and inlet (222), which is used to adjust the level of liquid sample within the at least one reservoir to a desired level.

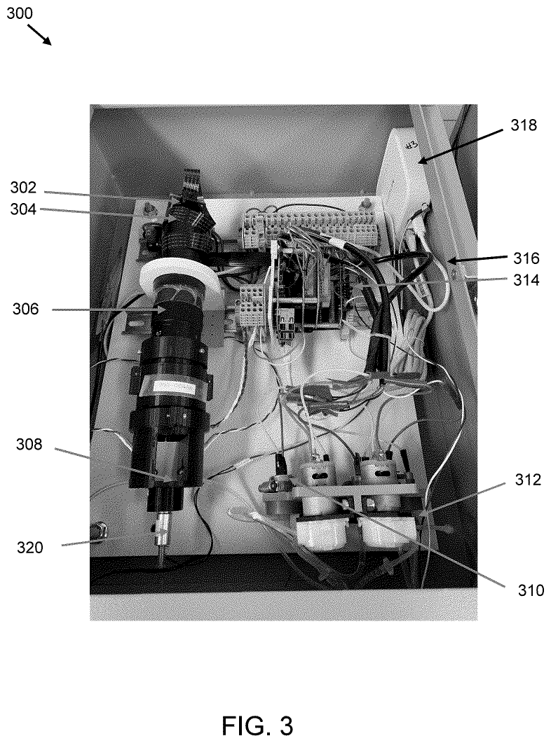

FIG. 3 illustrates an example of a detection unit as described herein. The detection unit (300) comprises an image capturing device comprising a Raspberry Pi (302) and camera (304), microscopic lens (306), a field of view (308), and at least one magnet or magnetizable member (320) for engaging at least one component within the field of view (308) of the image capturing device. The detection unit further comprises: operable valves (310); and a pump (312) to control the movement of liquid sample. The detection unit further comprises: an automation system (316) comprising a processor (314); and a modem (318) for wireless control and/or transmission of data.

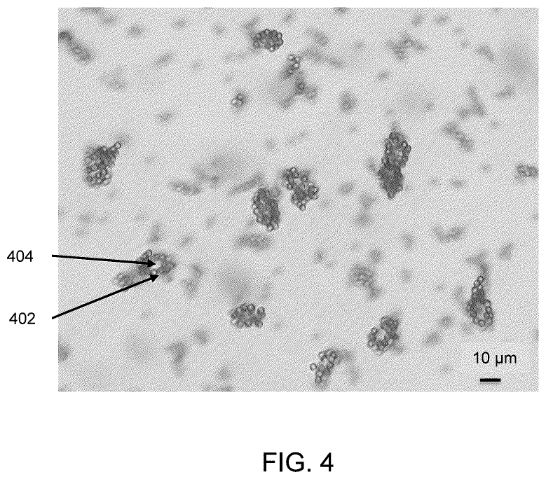

FIG. 4 illustrates an example of an image of antibody coated beads (402) conjugated to a spore (404), as disclosed herein. Spores were mixed with antibody coated beads and incubated at room temperature for more than 4 hours. After incubation, the mixture was examined under a compound microscope and the image was taken with a camera.

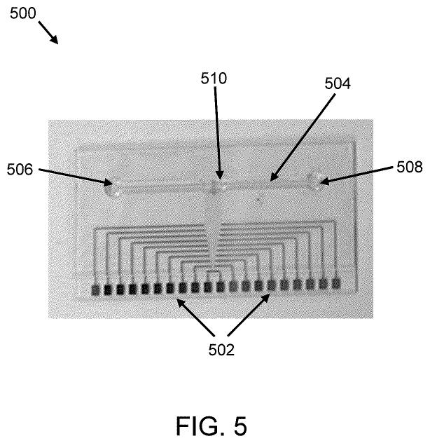

FIG. 5 illustrates an example of a biosensor as described herein. The biosensor (500) comprises: a support structure (not shown); 20 electrodes (502) coupled to the support structure; and an impedance measurement circuit (now shown) coupled to the electrodes (502). The biosensor (500) further comprises a flow channel (504) having an inlet (506) and an outlet (508), and a measurement point (510), which is a position along the flow channel where changes in impedance are measured as spores flow through the position.

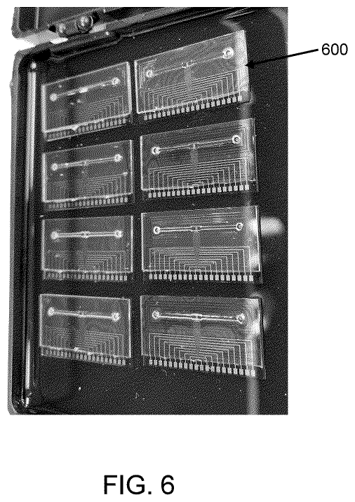

FIG. 6 illustrates an example of eight biosensors (600) connected together in series.

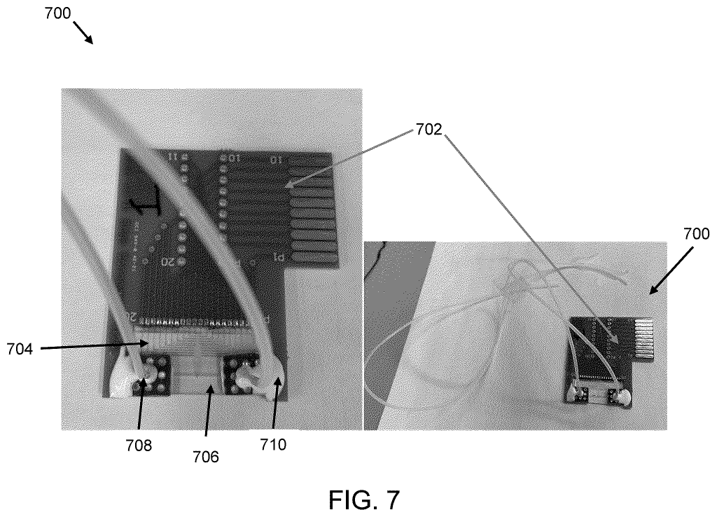

FIG. 7 illustrates an example of a biosensor as disclosed herein. The biosensor (700) comprises: an electric circuit board support structure (702); electrodes (704) coupled to the support structure; and an impedance measurement circuit (not shown) coupled to the electrodes (704). The biosensor (700) further comprises a flow channel (706) having an inlet (708) and an outlet (710) for moving liquid sample to and from the flow channel (706).

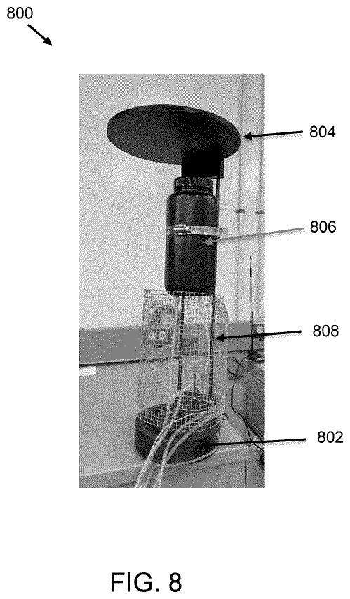

FIG. 8 illustrates an example of a sample trap device as described herein. The sample trap device (800) comprises: two reservoirs (802); a weather shield (804); a liquid storage chamber (806); and a net (808).

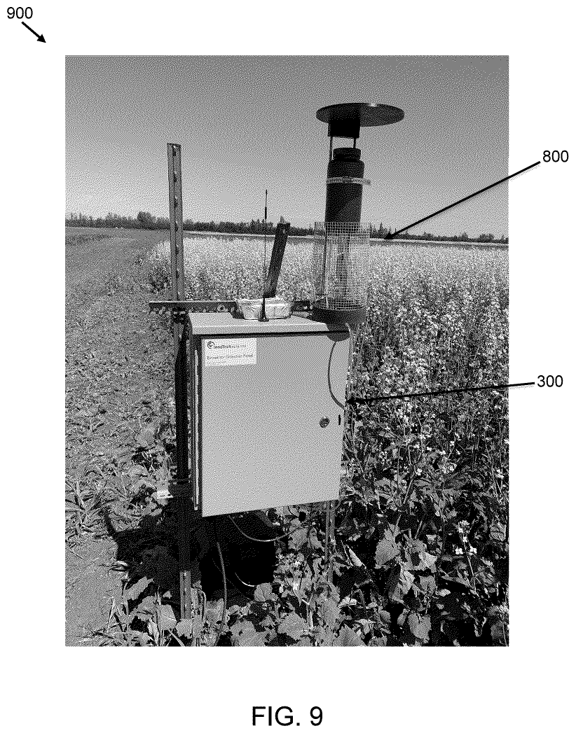

FIG. 9. illustrates an example of a sample trap device as described herein. The sample trap device (900) comprises: the sample trap device as shown in FIG. 8 coupled to the detection unit as shown in FIG. 3.

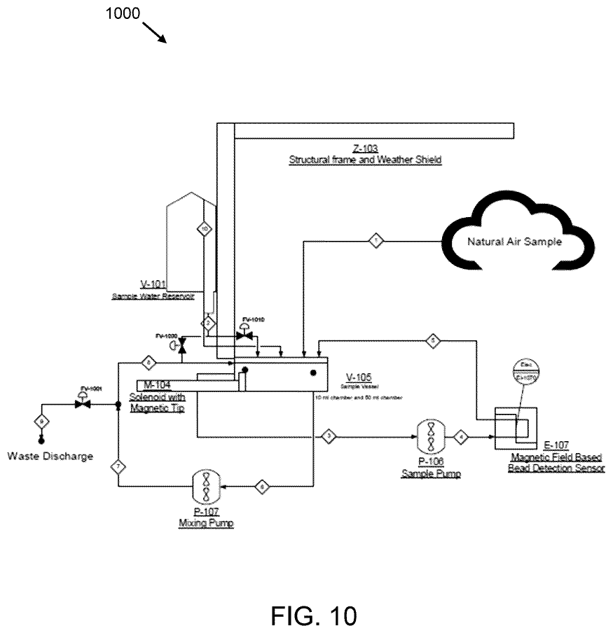

FIG. 10 depicts a circuit schematic of an example of a sample trap device as described herein. The biosensor remains turned off for most of the time. Within the system an independently powered clock device (real time clock) enables the power and timely operation of the entire biosensor. Once per hour, the system turns on to top up the sample reservoir by actuating a valve. Once per day the system performs an analysis sequence. A pump and valve enable a mixing sequence to agitate the sample reservoir so the microbeads can circulate with the spores. After this, the second pump moves the mixed sample through the flew cell for analysis. Once the flow analysis is complete, the data is prepared for transmission. The cellular modem is activated and once the network link is established a time synchronization is performed, and the data transmitted to an e-mail account. All the field units transmit to the same account. With the transmission concluded, the system fully shuts down and awaits the next hour where the top up sequence occurs.

### EXAMPLES

Table 1 shows data collected from a herein disclosed biosensor unit deployed in the summer of 2022.

TABLE 1
Spore Number Collected from the
Biosensor (900-00486) Deployed in Vegreville

| Date | Biosensor Spore count |
|---|---|
| Jul. 7, 2022 | |
| Jul. 8, 2022 | 3 |
| Jul. 9, 2022 | 14 |
| Jul. 10, 2022 | 16 |
| Jul. 11, 2022 | 18 |
| Jul. 12, 2022 | 31 |
| Jul. 13, 2022 | |
| Jul. 14, 2022 | |
| Jul. 15, 2022 | 78 |
| Jul. 16, 2022 | |
| Jul. 17, 2022 | |
| Jul. 18, 2022 | 62 |
| Jul. 19, 2022 | 83 |
| Jul. 20, 2022 | 78 |
| Jul. 21, 2022 | 113 |
| Jul. 22, 2022 | 96 |
| Jul. 23, 2022 | 127 |
| Jul. 24, 2022 | |
| Jul. 25, 2022 | 119 |
| Jul. 26, 2022 | 117 |
| Jul. 27, 2022 | 125 |
| Jul. 28, 2022 | 169 |
| Jul. 29, 2022 | 142 |
| Jul. 30, 2022 | 149 |
| Jul. 31, 2022 | 53 |
| Aug. 1, 2022 | 175 |
| Aug. 2, 2022 | 87 |
| Aug. 3, 2022 | 124 |
| Aug. 4, 2022 | 94 |
| Aug. 5, 2022 | 110 |
| Aug. 6, 2022 | 114 |

1. A sample trap device, comprising:
at least one reservoir having at least one outlet; and
at least one magnet or magnetizable member switchable between an activated magnetic field state and a deactivated magnetic field state, the magnet or magnetizable member operably coupleable to the reservoir to magnetically engage at least one component within the reservoir,
wherein the at least one outlet is in fluid communication with a detection unit.

2. (canceled)

3. The sample trap device of claim 1, wherein the at least one magnet or magnetizable member is coupleable to a side wall of the at least one reservoir and/or bottom of the at least one reservoir.

4. (canceled)

5. The sample trap device of claim 1, wherein at least two portions of the interior surface of the at least one reservoir slant downwards towards one another and meeting along the approximate centerline of the at least one reservoir.

6. (canceled)

7. The sample trap device of claim 1, wherein at least a portion of the side wall of the reservoir has an angle of inclination that is greater than about 90 degrees relative to the bottom of the reservoir and the at least one magnet or magnetizable member is operably coupleable to magnetically engage the at least one component on the portion of the side wall.

8. (canceled)

9. (canceled)

10. The sample trap device claim 1, wherein the at least one reservoir comprises another inlet located at the portion of the side wall and is in fluid communication with an outlet of the reservoir and/or with a liquid storage chamber.

11. (canceled)

12. The sample trap device of claim 1, wherein the device comprises two reservoirs, wherein the at least one reservoir and the other reservoir are in fluid communication with one another, and wherein a weir is located at least partially within the fluid communication path.

13. (canceled)

14. (canceled)

15. (canceled)

16. (canceled)

17. (canceled)

18. (canceled)

19. The sample trap device of claim 1, wherein the detection unit comprises an image capturing device and/or a biosensor.

20. (canceled)

21. The sample trap device of claim 19, wherein the image capturing device comprises a least one magnet or magnetizable member switchable between an activated magnetic field state and a deactivated magnetic field state, the at least one magnet or magnetizable member operably coupleable to the image capturing device to magnetically engage the at least one component, and wherein the at least one magnet or magnetizable member magnetically engages the at least one component within the field of view of the image capturing device.

22. (canceled)

23. (canceled)

24. (canceled)

25. The sample trap device of claim 19, wherein the biosensor comprises: a support structure; at least two electrodes coupled to the support structure; an impedance measurement circuit coupled to the at least two electrodes, and a flow channel in fluid communication with the at least one outlet, wherein the at least two electrodes are in fluid communication with at least a portion of the flow channel.

26. (canceled)

27. (canceled)

28. (canceled)

29. (canceled)

30. (canceled)

31. (canceled)

32. (canceled)

33. The sample trap device of claim 1, wherein the device is in communication with a processor configured to switch the magnet or magnetizable member between an activated magnetic field state and a deactivated magnetic field state; control the movement of fluid to and from the at least one reservoir, for example, feeding a sample from the at least one reservoir to the detection unit; and/or operating the detection unit.

34. (canceled)

35. (canceled)

36. The sample trap device of claim 1, wherein the sample comprises at least one fungal plant pathogen that infects a canola plant, a wheat plant, a barley plant, a corn plant, a rice plant, a millet plant, a sorghum plant, or a combination thereof, or Fusarium graminearum, F.avenaceum, F. poae, F.sporotrichioedes, Puccinia spp., Puccinia triticina, P. recondite, P. striiformis, Erysiphe graminis f.sp. tritici, Glomerella graminicola (anamorph Colletotrichum graminicola), Pyrenophora tritici-repentis (telomorph) and Drechslera tritici-repentis, Pyrenophora trichostoma, Urocystis agropyri, Sclerotinia borealis, Septoria spp., Stagnospora spp. Parastagonospora nodorum, Pyrenophora spp. teres tares, Pyrenophora teres maculate, Claviceps purpurea, Alternaria spp., Heminthosporium spp, Pseudocercosporella herpotrichoides, Glomerella graminicola (anamorphic), Colletotrichum graminicola, Fusarium verticillioides, Gibberella zeae, Aspergillus flavus, A. parasiticus, Lasiodiplodia theobromae, Physoderma maydis, Exserohilum turcicum, Cochliobolus heterostrophus, Cercospora zeae-maydis and Cercospora zeinaor, Cochliobolus carbonum, Stenocarpella maydis, Puccinia polysora, Magnaporthe oryzae, Cochliobolus miyabeanus, Ascochyta oryzae, Drechslera gigantean, Microdochium albescens, Cercospora oryzae, Puccinia graminis f.sp. oryzae, Uromyces coronatus, Ramularia oryzae, Bipolaris setariae, Cercospora penniseti, Curvularia penniseti, Dactuliophora elongata, Drechslera dematioidea, Claviceps fusiformis, Exserohilum rostratum, Beniowskia sphaeroidea, Myrothecium roridum, Phyllosticta penicillariae, Pyricularia grisea, Puccinia substriata, Moesziomyces penicillariae, Sclerotium rolfsii, Gleocercospora sorghi, Sarocladium strictum (syns Acremonium strictum), Cephalosporium acremonium, Macrophomina phaseolina, Claviceps africana, Sphacelia sorghi, Fusarium spp. moniliforme (syn. Gibberella fujikuroi), F. thapsinum (syn. G. thapsina), Aspergillus spp., Fusarium andiyazi, F. nygamai, Penicillium spp., Cercospora sorghi, Passalora fusimaculans (syn. Cercospora fusimaculans), Setosphaeria turcica (syns. Exserohilum turcicum, Helminthosporium spp. turcicum), Periconia circinata, Ramulispora sorghicola, Gibberella fujikuroi (syns. Fusarium moniliforme var. subglutinans G. fujikuroi var. subglutinans, G. intermedia, F. proliferatum), Ascochyta sorghi, Puccinia purpurea, Sclerotium rolfsii (syn. Athelia rolfsii), Ramulispora sorghi, Phyllachora sacchari, Bipolaris sorghicola (syns. B. cookei, Helminthosporium cookei.), Gloeocercospora sorghi, or any combination thereof.

37. (canceled)

38. A method of detecting the presence of and/or the amount of at least one fungal plant pathogen in a sample, comprising:
contacting the at least one fungal plant pathogen with at least one biological component that specifically binds to the at least one fungal plant pathogen in a liquid sample;
detecting, such as imaging and/or biosensing, at least a portion of the liquid sample; and
correlating the detection with the presence and/or the amount of the at least one fungal plant pathogen in the liquid sample.

39. The method of claim 38, further comprising concentrating the liquid sample before detecting at least a portion of the liquid sample by attracting the at least one biological component bound to the at least one fungal plant pathogen to a portion of the sample, wherein the attracting is performed using a magnet or magnetizable member switchable between an activated magnetic field state and a deactivated magnetic field state.

40. (canceled)

41. (canceled)

42. The method of claim 38, further comprising washing the liquid sample before detecting at least a portion of the liquid sample by attracting the at least one biological component bound to the at least one fungal plant pathogen to a portion of the sample and adding another portion of the sample and/or liquid from a storage chamber to the portion of the sample, wherein the attracting is performed using a magnet or magnetizable member switchable between an activated magnetic field state and a deactivated magnetic field state.

43. (canceled)

44. (canceled)

45. The method of claim 38, wherein the biological component is an antibody or fragment thereof conjugated to a magnetic bead.

46. (canceled)

47. The method of claim 38, wherein the contacting; imaging; detecting; and/or correlating is automated.

48. (canceled)

49. (canceled)

50. The method of claim 38, wherein the at least one fungal plant pathogen infects a canola plant, a wheat plant, a barley plant, a corn plant, a rice plant, a millet plant, a sorghum plant, or a combination thereof, or Fusarium graminearum, F.avenaceum, F. poae, F.sporotrichioedes, Puccinia spp., Puccinia triticina, P. recondite, P. striiformis, Erysiphe graminis f.sp. tritici, Glomerella graminicola (anamorph Colletotrichum graminicola), Pyrenophora tritici-repentis (telomorph) and Drechslera tritici-repentis, Pyrenophora trichostoma, Urocystis agropyri, Sclerotinia borealis, Septoria spp., Stagnospora spp. Parastagonospora nodorum, Pyrenophora spp. teres tares, Pyrenophora teres maculate, Claviceps purpurea, Alternaria spp., Heminthosporium spp, Pseudocercosporella herpotrichoides, Glomerella graminicola (anamorphic), Colletotrichum graminicola, Fusarium verticillioides, Gibberella zeae, Aspergillus flavus, A. parasiticus, Lasiodiplodia theobromae, Physoderma maydis, Exserohilum turcicum, Cochliobolus heterostrophus, Cercospora zeae-maydis and Cercospora zeinaor, Cochliobolus carbonum, Stenocarpella maydis, Puccinia polysora, Magnaporthe oryzae, Cochliobolus miyabeanus, Ascochyta oryzae, Drechslera gigantean, Microdochium albescens, Cercospora oryzae, Puccinia graminis f.sp. oryzae, Uromyces coronatus, Ramularia oryzae, Bipolaris setariae, Cercospora penniseti, Curvularia penniseti, Dactuliophora elongata, Drechslera dematioidea, Claviceps fusiformis, Exserohilum rostratum, Beniowskia sphaeroidea, Myrothecium roridum, Phyllosticta penicillariae, Pyricularia grisea, Puccinia substriata, Moesziomyces penicillariae, Sclerotium rolfsii, Gleocercospora sorghi, Sarocladium strictum (syns Acremonium strictum), Cephalosporium acremonium, Macrophomina phaseolina, Claviceps africana, Sphacelia sorghi, Fusarium spp. moniliforme (syn. Gibberella fujikuroi), F. thapsinum (syn. G. thapsina), Aspergillus spp., Fusarium andiyazi, F. nygamai, Penicillium spp., Cercospora sorghi, Passalora fusimaculans (syn. Cercospora fusimaculans), Setosphaeria turcica (syns. Exserohilum turcicum, Helminthosporium spp. turcicum), Periconia circinata, Ramulispora sorghicola, Gibberella fujikuroi (syns. Fusarium moniliforme var. subglutinans G. fujikuroi var. subglutinans, G. intermedia, F. proliferatum), Ascochyta sorghi, Puccinia purpurea, Sclerotium rolfsii (syn. Athelia rolfsii), Ramulispora sorghi, Phyllachora sacchari, Bipolaris sorghicola (syns. B. cookei, Helminthosporium cookei.), Gloeocercospora sorghi, or any combination thereof.

51. (canceled)

52. The method of claim 38, wherein the detecting comprises imaging at least a portion of the liquid sample and/or biosensing at least a portion of the liquid sample.

53. (canceled)

54. (canceled)

55. The method of claim 52, further comprising concentrating the liquid sample within the field of view of the image capturing device using a magnet or magnetizable member switchable between an activated magnetic field state and a deactivated magnetic field state.

56. (canceled)

57. (canceled)

58. The method of claim 52, wherein biosensing comprises measuring the impedance of at least a portion of the liquid sample, comprising:
measuring the impedance of the at least a portion of the liquid sample; and
correlating the change of the measured impedance and a reference impedance with the presence and/or the amount of the at least one fungal plant pathogen in the liquid sample.

59. (canceled)

60. (canceled)

61. (canceled)

62. (canceled)
